(19) United States
(12) Patent Application Publication
(10) Pub. No.: US 2024/0100479 A1
(43) Pub. Date: Mar. 28, 2024

(54) IN-SITU ENRICHMENT AND ANALYTICAL METHOD FOR THE Hg(II) ISOTOPE IN AQUEOUS PHASE

(71) Applicant: INSTITUTE OF GEOCHEMISTRY CHINESE ACADEMY OF SCIENCES, Guiyang City (CN)

(72) Inventors: Heng Yao, Guiyang City (CN); Hongqian Yin, Guiyang City (CN); Xinbin Feng, Guiyang City (CN); Jun Luo, Guiyang City (CN)

(21) Appl. No.: 18/139,425

(22) Filed: Apr. 26, 2023

(30) Foreign Application Priority Data
Sep. 22, 2022 (CN) 202211159896.9

(51) Int. Cl. B01D 59/26; C22B 43/00; G01N 21/31; G01N 33/18

(52) U.S. Cl. CPC B01D 59/26; C22B 43/00; G01N 21/31; G01N 33/1813; B01D 2259/40

(57) ABSTRACT

The present disclosure relates to analysis of Hg(II) isotopes in the aqueous phase, and provides an in-situ enrichment method and an analytical method for Hg(II) isotopes in the aqueous phase. The in-situ enrichment method includes the following steps: conducting adsorption in the water sample by a diffusive gradient in thin-films (DGT) device to obtain an Hg(II)-adsorbed DGT device; where a binding gel of the DGT device is an NSBA gel; conducting elution on the NSBA gel in the Hg(II)-adsorbed DGT device to acquire an Hg(II)-containing eluate with a mercury concentration higher than or equal to 0.5 ng/mL; where the elution is carried out with an eluent of reverse aqua regia. In the in-situ enrichment method, the NSBA-DGT device only needs to be placed in the water sample to be investigated to perform in-situ Hg(II) adsorption without direct grab sampling.

### CROSS REFERENCE TO RELATED APPLICATION

This patent application claims the benefit and priority of Chinese Patent Application No. 202211159896.9, filed with the China National Intellectual Property Administration on Sep. 22, 2022, the disclosure of which is incorporated by reference herein in its entirety as part of the present application.

### TECHNICAL FIELD

The present disclosure relates to the technical field of Hg(II) isotope determination in the aqueous phase, and in particular describes an in-situ enrichment and analytical method for the Hg(II) isotope in the aqueous phase.

### BACKGROUND

Mercury is recognized as a global pollutant of concern due to its harmful effect on the natural environment, amplified with the extent of its proliferation. Notably, mercury is known for its ease of transportation through the atmosphere and deposition into terrestrial and aquatic ecosystems. Once being deposited into the terrestrial and aquatic ecosystems, Hg is transformed by certain anaerobic microbes to the more toxic methylmercury (MeHg), which subsequently accumulates in the food chain, posing a threat to human health and the environment. The free Hg²⁺ and its labile Hg(II) complexes (together referred to as Hg(II)) are the most common forms of Hg in the aqueous phase as well as the major species for Hg methylation. The Hg(II) plays a crucial role in aquatic ecosystems due to its elevated activity and bioavailability. Mercury isotopes are a powerful tracing tool to help better understanding of the biogeochemical cycling of various Hg species in the aquatic ecosystems.

Currently, accurately tracing the source and transport of Hg in the aquatic ecosystems, faces significant challenges in terms of mercury isotope technology, mainly including: traditional sampling procedures are complex and cumbersome, with stringent sampling and storage requirements. This may lead to sample contamination during collection, transportation and storage. In addition, the physical and chemical properties of the sample (e.g. pH and redox conditions) are susceptible to change. These factors may cause significant uncertainty in the determination of sample concentrations and isotopic compositions at a later stage, leading to large errors in the assay results.

### SUMMARY

In the light of this, an objective of the present disclosure is to provide an in-situ enrichment and analytical method for Hg(II) isotopes in the aqueous phase. The in-situ enrichment method minimizes the potential for contamination during the transport of the water sample to be measured and provides high accuracy in the subsequent detection of Hg(II) isotopes in the aqueous phase.

To achieve the beforementioned objective of the present disclosure, the present disclosure provides the following technical solutions.

The present disclosure provides an in-situ enrichment method for Hg(II) isotopes in the aqueous phase, including the following steps:
(1) conducting adsorption on the water sample by a diffusive gradient in thin-films (DGT) device to obtain an Hg(II)-adsorbed DGT device; where a binding gel of the DGT device is an NSBA gel; and
(2) conducting elution on the NSBA gel in the Hg(II)-adsorbed DGT device obtained in step (1) to obtain an Hg(II)-containing eluate with a mercury concentration of higher than or equal to 0.5 ng/mL; where
the elution is carried out with reverse aqua regia as an eluent; and
the NSBA gel is a polyacrylamide hydrogel film that is internally provided with an SBA-15 mesoporous silicon material double-modified by thiol and amino groups at a molar ratio of (4-6):1.

Preferably, the time of adsorption is longer than or equal to 24 hours.

Preferably, the in-situ enrichment method further includes performing enrichment when the eluate has the mercury concentration of lower than 0.5 ng/mL; the enrichment is conducted by the first enrichment method or the second enrichment method; the first enrichment method includes: reducing the Hg(II) by adding a reducing agent to the eluent to obtain a reduction system; blowing nitrogen into the reduction system to reverse-enrich reduced Hg(0) into diluted reverse aqua regia with a volume percentage of 40%, to obtain an Hg(II)-containing eluate that meets the criteria for MC-ICP-MS analysis; where the reducing agent is a SnCl₂ aqueous solution with a concentration of 0.2 g/mL; and the nitrogen is blown at a flow rate of lower than or equal to 200 mL/min for 2 h.

Preferably, the second enrichment method includes: conducting elution a plurality of the NSBA gels in the Hg(II)-adsorbed DGT device, and combining obtained eluates to gain an Hg(II)-containing eluate that meets the criteria for MC-ICP-MS analysis.

Preferably, the Hg(II)-containing eluate has an acidity of higher than or equal to 40%.

The present disclosure further provides an analytical method for the Hg(II) isotope in the aqueous phase, including the following steps:
conducting in-situ enrichment on the water sample by the in-situ enrichment method to obtain an Hg(II)-containing eluate;
analyzing the Hg(II)-containing eluate by multi-collector inductively coupled plasma-mass spectrometry (MC-ICP-MS) to obtain the measured values of δ²⁰²Hg, Δ¹⁹⁹Hg, and Δ²⁰¹Hg; and
conducting correction on the measured value of δ²⁰²Hg to obtain an actual value of δ²⁰²Hg of the water to be tested; where
the correction has a correction value of −0.18‰.

Preferably, quality control of the water sample is conducted by MC-ICP-MS using GBW07405 and BCR-482 solid standard materials together with NIST SRM 3133 and NIST SRM 8610 mercury isotope standard solutions as external standards as well as an NIST 997 T1 standard solution as the internal standard.

The present disclosure provides an in-situ enrichment method for Hg(II) isotopes in the aqueous phase, including the following steps: (1) conducting adsorption on the water sample by a DGT device to obtain an Hg(II)-adsorbed DGT device; where an binding gel of the DGT device is an NSBA gel; and (2) conducting elution on the NSBA gel in the Hg(II)-adsorbed DGT device obtained in step (1) to acquire an Hg(II)-containing eluate with mercury concentrations of higher than or equal to 0.5 ng/mL; where the elution is conducted with an eluent of reverse aqua regia; and the NSBA gel is a polyacrylamide hydrogel film that is internally provided with an SBA-15 mesoporous silicon material double-modified by thiol and amino groups at a molar ratio of (4-6):1. In the in-situ enrichment method, users place the DGT device and the NSBA gel in the water samples to conduct in-situ adsorption of Hg(II) without using additional samplers. Therefore, the method reduces the risk of contamination and enhances accuracy in subsequent detection of the Hg(II) isotope in the aqueous phase.

The present disclosure further provides an analytical method for the Hg(II) isotope in the aqueous phase, including the following steps: (1) conducting in-situ enrichment on the water sample by the in-situ enrichment method to obtain an Hg(II)-containing eluate; analyzing the Hg(II)-containing eluate with MC-ICP-MS to obtain the measured values of δ²⁰²Hg, Δ¹⁹⁹Hg, and Δ²⁰¹Hg; and conducting correction on the measured value of δ²⁰²Hg to obtain an actual value of δ²⁰²Hg of the water to be tested; where the correction has a correction value of −0.18‰. In the analytical method, the DGT device provided with the NSBA gel is placed in the water sample to conduct in-situ Hg(II) enrichment. Compared with traditional methods of transport of water samples, the new protocol reduces the risk of sample contamination and improves detection accuracy.

### BRIEF DESCRIPTION OF THE DRAWINGS

FIG. 1 shows an elution effect of an NSBA gel using aqua regia and reverse aqua regia.

### DETAILED DESCRIPTION OF THE EMBODIMENTS

The present disclosure provides an in-situ enrichment method for Hg(II) isotope analysis in the aqueous phase, including the following steps:
(1) conducting adsorption on the water sample by a DGT device to obtain an Hg(II)-adsorbed DGT device; where a binding gel of the DGT device is an NSBA gel; and
(2) conducting elution on the NSBA gel in the Hg(II)-adsorbed DGT device obtained in step (1) to acquire an Hg(II)-containing eluate with mercury concentrations higher than or equal to 0.5 ng/mL; where

the elution is conducted with an eluent of reverse aqua regia; and

the NSBA gel is a polyacrylamide hydrogel film that is internally provided with an SBA-15 mesoporous silicon material double-modified by thiol and amino groups at a molar ratio of (4-6):1.

In the present disclosure, the raw materials provided herein are all preferably commercially-available products unless otherwise specified.

In the present disclosure, adsorption is conducted on the water sample by a DGT device to obtain an Hg(II)-adsorbed DGT device; where an binding gel of the DGT device is an NSBA gel.

In the present disclosure, the NSBA gel is a polyacrylamide hydrogel film that is internally provided with an SBA-15 mesoporous silicon material double-modified by thiol and amino groups at a molar ratio of (4-6):1.

In the present disclosure, a preparation method of the NSBA gel includes preferably the following steps:
Step 1, preparation of an SBA-15 mesoporous silicon material double-modified by thiol and amino groups:

(1) in a conical flask, fully dissolving a polyethylene oxide-polypropylene oxide-polyethylene oxide triblock copolymer (P123) with 1.9 mol/L hydrochloric acid, raising the reaction temperature to 40° C., adding ethyl orthosilicate (TEOS) dropwise at 5% of the molarity of the contained silicon to P123, stirring for 60 min, and adding (3-mercaptopropyl) trimethoxysilane (MPTMS) dropwise, where TEOS and MPTMS are added dropwise at a molar ratio of x=MPTMS/((TEOS+MPTMS)), x=0.2; after stirring for 20 h, transferring the obtained mixed solution to an autoclave, aging at 100° C. for 24 h, and subjecting an obtained product to cooling, suction filtration, washing, and drying;
(2) conducting vacuum dehydration on the material obtained in step (1), transferring into a round-bottomed flask, adding toluene and (3-aminopropyl) trimethoxysilane (APTMS), and stirring at 110° C. for 24 h; washing the obtained product 2 to 3 times with toluene, absolute ethanol, and deionized water in sequence, and drying in vacuum; and
(3) adding the material obtained in step (2) and 95% ethanol into a round-bottomed flask, heating and stirring to reflux for 24 h; and subjecting the obtained reaction product to cooling, suction filtration, washing, and drying in vacuum to obtain an SBA-15 mesoporous silicon heavy metal adsorbent double-modified by thiol and amino groups;
Step 2, mixing a cross-linking agent DGT gel cross-linker with a concentration of 2% purchased from DGT Research Co., Ltd. (UK), MQ water, and an acrylamide solution produced by GE Healthcare at a volume ratio of 15:47.5:37.5, to obtain a glue-making solution, where the acrylamide solution has a concentration of 40%; and storing the glue-making solution in a 4° C. refrigerator for future use;
Step 3, fully grinding the SBA-15 mesoporous silicon material double-modified by thiol and amino groups prepared in step 1 and mixing with the glue-making solution prepared in step 2 at a mass-to-volume ratio of 1:30, to obtain a mixed glue-making solution, and performing sonication for 3 min to prepare a mixed solution;
Step 4, adding an ammonium persulfate solution at a concentration of 10% by mass to 1/150 of the volume of the polyacrylamide solution and a tetramethyldiethylamine solution at 1/800 of the volume of the polyacrylamide solution into the mixed solution prepared in step 3, and mixing evenly to obtain a mixed solution; and
Step 5, injecting the mixed solution prepared in step 4 into the aperture of two glass plates sandwiched with a 0.25 mm thick U-shaped Teflon sheet at 5 mL/min, and removing air bubbles; transferring the obtained product horizontally to an oven at 42° C. for 40 min; after the solution gel in the glass plate became a film, soaking the film in pure water for 24 h, during which time the membrane is washed 4 times with pure water to finish the preparation of NSBA adsorption membrane.

In the present disclosure, the adsorption is conducted for preferably longer than or equal to 24 h.

In the present disclosure, a shell, a filter membrane, and a diffusion layer of the DGT device each are purchased from Nanjing Vision Environmental Science & Technology Ltd. The filter membrane of the DGT device is preferably prepared with polyethersulfone (PES), and has a pore size of preferably 0.45 μm and a thickness of preferably 0.14 mm. The diffusion layer of the DGT device is preferably prepared by agarose gel, and has a thickness of preferably 0.8 mm. The DGT device has other structures consistent with the prior art.

In the present disclosure, elution is conducted on the NSBA gel in the Hg(II)-adsorbed DGT device to obtain an Hg(II)-containing eluate with a mercury concentration of higher than or equal to 0.5 ng/mL.

In the present disclosure, before elution, the NSBA gel in the Hg(II)-adsorbed DGT device is preferably rinsed. The rinsing is conducted with a reagent including preferably deionized water. There is no special limitation on rinsing time and frequency, as long as the impurities in the NSBA gel in the Hg(II)-adsorbed DGT device can be removed.

In the present disclosure, the elution is conducted with an eluent of reverse aqua regia. A volume of the eluent and an area of the NSBA gel have a ratio of preferably 5 mL: 3.14 cm 2. The elution is conducted via preferably vibration for preferably 8 h to 12 h.

In the present disclosure, the Hg(II)-containing eluate has an acidity of preferably higher than or equal to 40%, more preferably 40%.

In the present disclosure, the Hg(II) eluate has a mercury concentration of higher than or equal to 0.5 ng/mL. When the eluate has a mercury concentration of lower than 0.5 ng/mL, the in-situ enrichment method further includes preferably conducting enrichment; the enrichment includes preferably the first enrichment method or the second enrichment method.

In the present disclosure, the first enrichment method preferably includes: reducing the Hg(II) by adding a reducing agent to the eluent to obtain a reduction system; blowing nitrogen into the reduction system to reverse-enrich reduced Hg(0) into diluted reverse aqua regia with a volume percentage of 40%, to obtain an Hg(II)-containing eluate that meets the criteria for MC-ICP-MS analysis. The diluted reverse aqua regia with a volume percentage of 40% is a solution obtained by mixing the reverse aqua regia and water according to a volume ratio of 4:6. The reducing agent is preferably a SnCl₂ aqueous solution with a concentration of 0.2 g/mL; and there is no special limitation on an amount of the reducing agent, as long as Hg(II) can be completely converted into Hg(0). The nitrogen is blown at preferably lower than or equal to 200 mL/min, more preferably 100 mL/min to 200 mL/min for preferably 2 h.

In the present disclosure, the second enrichment method preferably includes: conducting elution a plurality of the NSBA gel in the Hg(II)-adsorbed DGT device, and combining obtained eluates to gain an Hg(II)-containing eluate which meets the criteria for MC-ICP-MS analysis.

The present disclosure further provides an analytical method for Hg(II) isotopes in the aqueous phase, including the following steps:
conducting in-situ enrichment on the water sample using the in-situ enrichment method to obtain an Hg(II)-containing eluate;
analyzing the Hg(II)-containing eluate with MC-ICP-MS to obtain measured values of δ²⁰²Hg, Δ¹⁹⁹Hg and Δ²⁰¹Hg; and
conducting correction on the measured value of δ²⁰²Hg to obtain an actual value of δ²⁰²Hg of the water to be tested; where
the correction has a correction value of −0.18‰.

In the present disclosure, in-situ enrichment is carried out on the water sample using the in-situ enrichment method to obtain an Hg(II)-containing eluate. The parameters of the in-situ enrichment are preferably consistent with the beforementioned technical solutions, and are not repeated here.

In the present disclosure, the Hg(II)-containing eluate is analyzed with MC-ICP-MS to obtain the measured values of δ²⁰²Hg, Δ¹⁹⁹Hg, and Δ²⁰¹Hg.

In the present disclosure, quality control of the water sample is preferably conducted with MC-ICP-MS using GBW07405 and BCR-482 solid standard materials together with NIST SRM 3133 and NIST SRM 8610 mercury isotope standard solutions as external standards as well as an NIST 997 T1 standard solution as the internal standard.

In the present disclosure, there is no special limitation on detection parameters of MC-ICP-MS, and detection parameters well known to those skilled in the art can be used.

In the present disclosure, correction is conducted on the measured value of δ²⁰²Hg to obtain an actual value of δ²⁰²Hg of the water to be tested.

In the present disclosure, the correction has a correction value of preferably −0.18‰.

In the present disclosure, the Hg-MDF/MIF value is preferably obtained through the following steps:
(a) The DGT device (with NSBA gel) is placed into the 100 mL standard solution containing 0.01 mol/L NaCl and ˜60 ng/mL NIST SRM 3133 isotope mercury at different temperatures (15° C., 25° C., and 35° C.); a thermostatic oscillator has a shaking speed of 120 rpm; at different time points from 0 to 24 h, the DGT device is removed from the solution and the NSBA gel is removed from the DGT device, and then eluted with 5 mL of the reverse aqua regia for 8 h to 12 h to obtain eluates at different time points. In addition, 1% BrCl was added to residual solutions (remaining solutions after taking out the DGT device at different time points) to convert other forms of mercury into Hg(II), and then stored in a refrigerator at 4° C. for subsequent Hg(II) concentration and isotope analysis.

When mercury concentrations in the eluates at different time points do not reach the standard for isotope determination (not lower than 0.5 ng/mL) with MC-ICP-MS, the eluates at different time points can be reverse-enriched using a mercury enrichment system of cold vapor atomic fluorescence (CV-AFS); a specific method includes: putting 10 mL to 12 mL of an eluate to be enriched into a bubble bottle, and adding 5 mL of a SnCl₂ aqueous solution with a concentration of 0.2 g/mL to the bubble bottle to reduce Hg(II), such that Hg(II) in the eluate to be enriched is converted into Hg(0); and blowing nitrogen at 100 mL/min to 200 mL/min into the bubble bottle for 2 h, such that reduced Hg(0) can be reverse-enriched in 5 mL of diluted reverse aqua regia with a volume percentage of 40%.
(b) For quality control, GBW07405 and BCR-482 solid standard solutions were prepared, 0.5 ng/mL of an NIST SRM 3133 mercury isotope standard solution and an NIST SRM 8610 mercury isotope standard solution were accurately prepared, and a 20 ng/mL T1 (NIST 997) standard solution was prepared.
(c) The eluates at different time points are analyzed with an online mercury generation system in combination with MC-ICP-MS, to obtain measured values of δ²⁰²Hg, Δ¹⁹⁹Hg, and Δ²⁰¹Hg in the sample.

The proportion and isotopic values of mercury adsorbed to the DGT device at different temperatures are shown in Table 1.

TABLE 1
Proportion and isotopic values of mercury adsorbed to DGT at
different temperatures

| Temperature | | δ²⁰²Hg | | Δ¹⁹⁹Hg | | Δ²⁰¹Hg | |
|---|---|---|---|---|---|---|---|
| (° C.) | (1-f)_{Hg(II)} | (‰) | SD | (‰) | SD | (‰) | SD |
| 15 | 0-20% | −0.18 | 0.05 | −0.01 | 0.03 | −0.01 | 0.03 |
| 25 | 0-25% | −0.2 | 0.06 | −0.01 | 0.05 | 0.00 | 0.03 |
| 35 | 0-25% | −0.15 | 0.06 | 0.01 | 0.04 | 0.00 | 0.04 |

As shown in Table 1 that at 15° C., 25° C., and 35° C., compared with the reference values of δ²⁰²Hg and Δ¹⁹⁹Hg (˜0‰) in NIST SRM 3133 initial solution, during adsorption of mercury to DGT, a small Hg-MDF of about −0.2‰ in δ²⁰²Hg could occur during the experiments, however, it should be cautious that this value may vary due to the complex Hg speciation and matrices in natural waters. In contrast, no mass independent isotope fractionation (MIF) for odd-mass-number isotopes (˜0‰) were observed during the experiments at all temperatures, according to the consistent Δ¹⁹⁹Hg and Δ²⁰¹Hg observed in all experiments. This highlights the great potential to use DGT Δ¹⁹⁹Hg and Δ²⁰¹Hg values to monitor the MIF signature of Hg in natural waters.

The in-situ enrichment method and the analytical method for the Hg(II) isotope in the aqueous phase provided by the present disclosure are detailed below with reference to the examples, but these examples may not be understood as a limitation to the protection scope of the present disclosure.

### Example 1

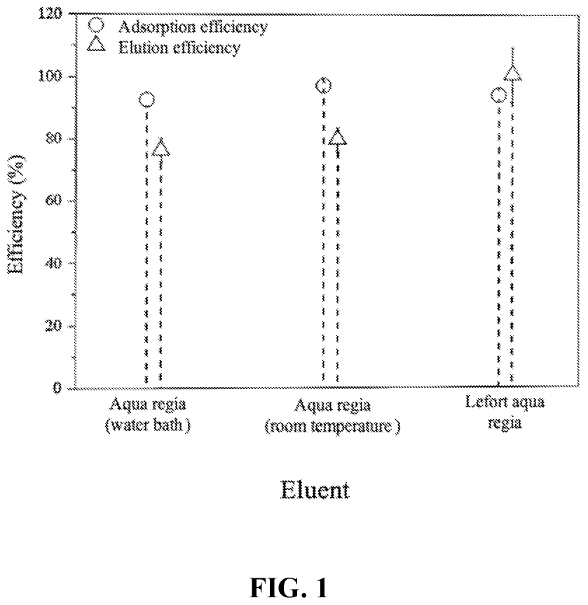

Selection of an Eluent
(I) An NSBA gel (3.14 cm 2, prepared via the aforementioned preparation method of an NSBA gel) was soaked into 30 mL of a solution to be investigated containing 0.01 mol/L NaCl and 1 ng/mL Hg(II) to perform adsorption for 24 h, to obtain an Hg(II)-adsorbed NSBA gel.
(II) The impurities on the surface of the Hg(II)-adsorbed NSBA gel obtained in step (I) were rinsed with ultrapure water, water on the surface of the Hg(II)-adsorbed NSBA gel was removed with lens tissue, and a rinsed Hg(II)-adsorbed NSBA gel was placed in a Teflon bottle containing 5 mL of aqua regia and 5 mL of reverse aqua regia and then eluted by continuous shaking. The elution with aqua regia was conducted by heating in a water bath for 3 h, or at a room temperature for 8 h, and the elution with reverse aqua regia was conducted at room temperature for 8 h to 12 h; such that three different eluates were obtained.
(III) 5 mL of ultrapure water was separately added to the three eluates obtained in step (II) for dilution, so as to obtain three different kinds of Hg(II)-containing eluates.
(IV) Using CV-AFS (Tekran 2500), mercury concentrations in the three Hg(II)-containing eluates obtained from step (III) and concentrations in the solution to be examined before and after adsorption by the NSBA gel were determined.
(V) The adsorption efficiency of the NSBA gel to Hg(II) and the elution efficiency with aqua regia and reverse aqua regia to Hg(II) were calculated from the data of step (IV), and results were shown in FIG. 1.

As shown in FIG. 1 that 5 mL of the reverse aqua regia had an elution efficiency of 100.75%±8.91% for Hg(II), while 5 mL of the aqua regia (both in water bath and room temperature) had an elution efficiency of 76.36% to 79.87% for Hg(II). Therefore, the reverse aqua regia was confirmed to be the eluent of the NSBA gel.

### Example 2

Indoor experimental verification
(1) The DGT device (with a NSBA gel, a filter membrane of a PES film with a pore size of 0.45 μm and a thickness of 0.14 mm, and a diffusion layer of an agarose gel film with a thickness of 0.8 mm) was placed into 100 mL of an solution containing 0.01 mol/L NaCl and ˜60 ng/mL NIST SRM 3133 isotope mercury standard at different temperatures (15° C., 25° C., and 35° C.); a thermostatic oscillator had a shaking speed of 120 rpm; at different time points from 0 to 24 h, the DGT device was removed from the solution and the NSBA gel was removed from the DGT device, and then eluted with 5 mL of the reverse aqua regia for 12 h to obtain eluates at different time points. In addition, 1% BrCl was added to residual solutions (remaining solutions after taking out the DGT device at different time points) to convert other forms of mercury into Hg(II), and then stored in a 4° C. refrigerator for storage, steps (III) and (IV) of Example 1 were repeated.

If mercury concentrations in the eluates at different time points did not reach the criterion for isotope determination (>0.5 ng/mL) with MC-ICP-MS, the eluates at different time points could be reverse-enriched using a mercury enrichment system of CV-AFS; a specific method included: 10 mL of an eluate to be enriched was put into a bubble bottle, and 5 mL of a SnCl₂ aqueous solution with a concentration of 0.2 g/mL was added to the bubble bottle to reduce Hg(II), such that Hg(II) in the eluate to be enriched was converted into Hg(0); and nitrogen was blown at 100 mL/min to 200 mL/min into the bubble bottle for 2 h, such that reduced Hg(0) could be reverse-enriched in 5 mL of diluted reverse aqua regia with a volume percentage of 40%.
(2) GBW07405 and BCR-482 solid standard solutions were prepared, 0.5 ng/mL of an NIST SRM 3133 mercury isotope standard solution and an NIST SRM 8610 mercury isotope standard solution were accurately prepared, and a 20 ng/mL T1 (NIST 997) standard solution was prepared for quality control.
(3) The eluates at different time points were detected by an online mercury generation system in combination with MC-ICP-MS, to obtain the measured values of δ²⁰²Hg, Δ¹⁹⁹Hg, and Δ²⁰¹Hg in the samples.

The instrumental stability of MC-ICP-MS was ensured via NIST SRM 3133 and three standard materials (internal and external standards). The specific results were shown in Table 2.

TABLE 2
Experimental data on the stability of the MC-ICP-MS instrument

| Name | n | δ²⁰²Hg | 2σ | Δ¹⁹⁹Hg | 2σ | Δ²⁰⁰Hg | 2σ | Δ²⁰¹Hg | 2σ |
|---|---|---|---|---|---|---|---|---|---|
| NIST-8610 | 16 | −0.53 | 0.07 | −0.03 | 0.07 | 0.01 | 0.05 | −0.03 | 0.08 |
| BCR-482 | 2 | −1.69 | 0.07 | −0.62 | 0.07 | 0.09 | 0.05 | −0.65 | 0.08 |
| GBW07405 | 2 | −1.76 | 0.07 | −0.31 | 0.07 | 0.03 | 0.05 | −0.30 | 0.08 |

Table 2 shows that the measurement results were consistent with the reference values, indicating that during the measurement of all samples, MC-ICP-MS was relatively stable during operation.

As shown in Table 1, Hg-MDF produced a negative offset of 0.2‰ during the DGT adsorption of mercury. Based on this, it should be cautioned that Hg-MDF values may vary due to the complex Hg speciation and matrices in natural waters. In contrast, no mass independent isotope fractionation (MIF) for odd-mass-number isotopes (˜0‰) were observed during the experiments at all temperatures, according to the consistent Δ¹⁹⁹Hg and Δ²⁰¹Hg observed in all experiments. This implied that the MIF value detected via DGT sampling could reflect the real MIF signal of the natural samples, thereby further evidencing the reliability of the method.

### Example 3

Field Application

The NSBA-DGT devices were placed into a paddy soil solution then removed after 14 days. Surface impurities of the devices were rinsed by ultrapure water on site, and the device was subjected to elution and detection in the laboratory. The elution and detection were the same as those mentioned in Examples 1 and 2, and the results were shown in Table 3.

TABLE 3
Isotopic composition of mercury in environmental
samples with DGT sampling.

| | δ²⁰²Hg | | Δ¹⁹⁹Hg | | Δ²⁰¹Hg | |
|---|---|---|---|---|---|---|
| DGT | (‰) | 2σ | (‰) | 2σ | (‰) | 2σ |
| DGT-1 | −2.17 | 0.07 | 0.11 | 0.07 | 0.06 | 0.08 |
| DGT-2 | −2.28 | 0.07 | 0.22 | 0.07 | −0.01 | 0.08 |

Table 3 shows that the combination of DGT and MC-ICP-MS could determine the mercury isotopic composition of Hg(II) in the aqueous phase of the paddy soil. As shown in Table 3, measured values of δ²⁰²Hg was −2.28‰ and −2.17‰, given that Hg-MDF can occur during the use of DGT and the extent of MDF may vary between laboratory and field conditions, we emphasize MIF signals, it can be seen that a slight mass-independent fractionation (MIF) was detected, and the measured values of Δ¹⁹⁹Hg was 0.11‰ and 0.22‰.

The beforementioned descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. An in-situ enrichment method for Hg(II) isotope analysis in an aqueous phase, comprising the following steps:
(1) conducting adsorption on a water sample by a diffusive gradient in thin-films (DGT) device to obtain an Hg(II)-adsorbed DGT device; wherein a binding gel of the DGT device is an NSBA gel; and
(2) conducting elution on the NSBA gel in the Hg(II)-adsorbed DGT device obtained in step (1) to acquire an Hg(II)-containing eluate with a mercury concentration of higher than or equal to 0.5 ng/mL; wherein:
the elution is carried out with an eluent of reverse aqua regia; and
the NSBA gel is a polyacrylamide hydrogel film that is internally provided with an SBA-15 mesoporous silicon material double-modified by thiol and amino groups at a molar ratio of (4-6):1.

2. The in-situ enrichment method according to claim 1, wherein the adsorption is conducted for longer than or equal to 24 h.

3. The in-situ enrichment method according to claim 1, further comprising the performance of the enrichment process when the eluate has the mercury concentration of lower than 0.5 ng/mL; the enrichment is conducted by a first enrichment method or a second enrichment method; the first enrichment method comprises: reducing the Hg(II) by adding a reducing agent to the eluent to obtain a reduction system; blowing nitrogen into the reduction system to reverse-enrich reduced Hg(0) into diluted reverse aqua regia with a volume percentage of 40%, to obtain an Hg(II)-containing eluate that meets the criteria for MC-ICP-MS analysis; wherein the reducing agent is a SnCl₂ aqueous solution with a concentration of 0.2 g/mL; and the nitrogen is blown at a flow rate of lower than or equal to 200 mL/min for 2 h.

4. The in-situ enrichment method according to claim 3, wherein the second enrichment method comprises: conducting elution on a plurality of the NSBA gels in the Hg(II)-adsorbed DGT device, and combining obtained eluates to gain an Hg(II)-containing eluate that meets the criteria for MC-ICP-MS analysis.

5. The in-situ enrichment method according to claim 1, wherein the Hg(II)-containing eluate has an acidity of higher than or equal to 40%.

6. An analytical method for Hg(II) isotopes in an aqueous phase, comprising the following steps:
conducting in-situ enrichment on a water sample by the in-situ enrichment method according to claim 1 to obtain an Hg(II)-containing eluate;
detecting the Hg(II)-containing eluate by multicollector inductively coupled plasma-mass spectrometry (MC-ICP-MS) to obtain measured values of δ²⁰²Hg, Δ¹⁹⁹Hg, and Δ²⁰¹Hg; and
conducting correction on the measured value of δ²⁰²Hg to obtain an actual value of δ²⁰²Hg of the water to be tested; wherein
the correction has a correction value of −0.18‰.

7. The analytical method according to claim 6, wherein the adsorption is conducted for longer than or equal to 24 h.

8. The analytical method according to claim 6, further comprising conducting enrichment when the eluate has a mercury concentration of lower than 0.5 ng/mL; the enrichment is conducted by the first enrichment method or the second enrichment method; the first enrichment method comprises: reducing the Hg(II) by adding a reducing agent to the eluent to obtain a reduction system; blowing nitrogen into the reduction system to reverse-enrich reduced Hg(0) into diluted reverse aqua regia with a volume percentage of 40%, to obtain an Hg(II)-containing eluate that meets the criteria for MC-ICP-MS analysis; wherein the reducing agent is a SnCl₂ aqueous solution with a concentration of 0.2 g/mL; and the nitrogen is blown at a flow rate of lower than or equal to 200 mL/min for 2 h.

9. The analytical method according to claim 8, wherein the second enrichment method comprises: conducting elution a plurality of the NSBA gels in the Hg(II)-adsorbed DGT device, and combining obtained eluates to obtain an Hg(II)-containing eluate that meets the criteria for MC-ICP-MS analysis.

10. The analytical method according to claim 6, wherein the Hg(II)-containing eluate has an acidity of higher than or equal to 40%.

11. The analytical method according to claim 6, wherein quality control of the water sample is conducted by MC-ICP-MS using GBW07405 and BCR-482 solid standard materials together with NIST SRM 3133 and NIST SRM 8610 mercury isotope standard solutions as external standards as well as an NIST 997 T1 standard solution as the internal standard.

12. The analytical method according to claim 7, wherein quality control of the water sample is conducted by MC-ICP-MS using GBW07405 and BCR-482 solid standard materials together with NIST SRM 3133 and NIST SRM 8610 mercury isotope standard solutions as external standards as well as an NIST 997 T1 standard solution as the internal standard.

13. The analytical method according to claim 8, wherein quality control of the water sample is conducted by MC-ICP-MS using GBW07405 and BCR-482 solid standard materials together with NIST SRM 3133 and NIST SRM 8610 mercury isotope standard solutions as external standards as well as an NIST 997 T1 standard solution as the internal standard.

14. The analytical method according to claim 9, wherein quality control of the water sample is conducted with MC-ICP-MS using GBW07405 and BCR-482 solid standard materials together with NIST SRM 3133 and NIST SRM 8610 mercury isotope standard solutions as external standards as well as an NIST 997 T1 standard solution as the internal standard.

15. The analytical method according to claim 10, wherein quality control of the water sample is conducted with MC-ICP-MS using GBW07405 and BCR-482 solid standard materials together with NIST SRM 3133 and NIST SRM 8610 mercury isotope standard solutions as external standards as well as an NIST 997 T1 standard solution as the internal standard.
